(19) United States
(12) Patent Application Publication
(10) Pub. No.: US 2026/0132481 A1
(43) Pub. Date: May 14, 2026

(54) BIOREACTOR SYSTEM TO PRODUCE METAL

(71) Applicant: ECOBIOME HOLDINGS LLC, THE WOODLANDS, TX (US)

(72) Inventor: MARC RODRIGUEZ, THE WOODLANDS, TX (US)

(73) Assignee: ECOBIOME HOLDINGS LLC, THE WOODLANDS, TX (US)

(21) Appl. No.: 18/704,577

(22) PCT Filed: Nov. 3, 2022
(86) PCT No.: PCT/US2022/079241

§ 371 Date: Apr. 25, 2024

Related U.S. Application Data
(60) Provisional application No. 63/275,058, filed on Nov. 3, 2021.

(51) Int. Cl. C22B 3/18; C12M 1/06; C12M 1/42; C22B 3/02; C22B 59/00

(52) U.S. Cl. CPC C22B 3/18; C12M 27/02; C12M 35/02; C22B 3/02; C22B 59/00

(57) ABSTRACT

A bioreactor stimulator is provided. A platform is operable to receive a metal sample of a desired metal. The metal sample is operable to interact with microbe and stimulate the microbe to recover the desired metal from selection material. An electrical conductor is electrically coupled to the platform. The electrical conductor and the metal sample are operable to receive and transmit electric pulses. The electrical conductor is operable to transmit the electric pulses to the selection material to stimulate the microbe in the selection material to recover the desired metal.

### FIELD

The present disclosure is directed to the field of metal extraction, production, and amplification. In at least one example, the present disclosure relates to stimulating microbe within a selection material to improve production of metal from the selection material.

### BACKGROUND

The price of gold and other precious metals continuously increase, lifted by robust demand and a shortage of precious metals supplies. Global demand for precious and rare earth metals is rising rapidly and supply is not growing quickly enough to match the increasing demand. Many nations, particularly those with developing economies, do not have reliable access to precious and rare earth metals. In addition, inefficiencies in the supply chain, from mining and refinement to distribution and usage, make the availability of precious and rare earth metals unpredictable.

For example, gold mine production totaled 3,531 tons in 2019, 1% lower than in 2018, according to the World Gold Council, which marked the first annual decline in production since 2008. The growth in mine supply is expected to slow or decline slightly in the coming years, as existing reserves are exhausted, and new major discoveries become increasingly rare.

### BRIEF DESCRIPTION OF THE DRAWINGS

Implementations of the present technology will now be described, by way of example only, with reference to the attached figures, wherein:

FIGS. 1A and 1B are schematic views of an exemplary bioreactor system;

FIG. 2A is a diagram of an exemplary container;

FIG. 2B is a diagram of an electrical connection between the container and a power supply;

FIG. 3 illustrates a top, isometric view of a platform and one or more input conduits;

FIG. 4 illustrates another top, isometric view of the platform and input conduits along with a frame to receive the container;

FIG. 5A is a diagram of an exemplary platform having a gold crystal structure shape to receive a gold metal sample;

FIG. 5B is a diagram of an exemplary platform having a metal crystal structure shape to receive a metal sample.

FIG. 6 is a schematic view of an exemplary bioreactor system with a bioreactor stimulator disposed in the container;

FIG. 7 is a schematic view of an exemplary bioreactor system with a bioreactor stimulator disposed in the inlet conduit;

FIG. 8 is a schematic view of an exemplary bioreactor system with a bioreactor stimulator disposed in the container; and

FIG. 9 is a flow chart of a method for stimulating microbe to improve production of metal from selection material according to the disclosure herein.

### DETAILED DESCRIPTION

It will be appreciated that for simplicity and clarity of illustration, where appropriate, reference numerals have been repeated among the different figures to indicate corresponding or analogous elements. In addition, while specific details are set forth in order to provide an understanding of the examples described herein, it will be appreciated by those skilled in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “communicatively coupled” is defined as connected, either directly or indirectly through intervening components, and the connections are not necessarily limited to physical connections, but are connections that accommodate the transfer of data between the so-described components. The connection can be such that the objects are permanently connected or releasably connected. The terms “comprising,” “including” and “having” are used interchangeably in this disclosure. The terms “comprising,” “including” and “having” mean to include, but not necessarily be limited to the things so described.

Where components are described as being “configured to” or “operable to” perform certain operations, such configuration or operation can be accomplished, for example, by designing electronic circuits or other hardware to perform the operation, by programming programmable electronic circuits (e.g., microprocessors, or other suitable electronic circuits) to perform the operation, or any combination thereof.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The present disclosure provides for improving production, extraction, amplification, and/or retrieval of metal disposed within selection material. Microbe is introduced into selection material that includes desired metal to be recovered. The microbe interacts with a metal sample to help the microbe recognize and be sensitized to the desired metal. Electric pulses are transmitted through the metal sample and an electrical conductor to the selection material to further stimulate the microbe in the selection material to recover the desired metal.

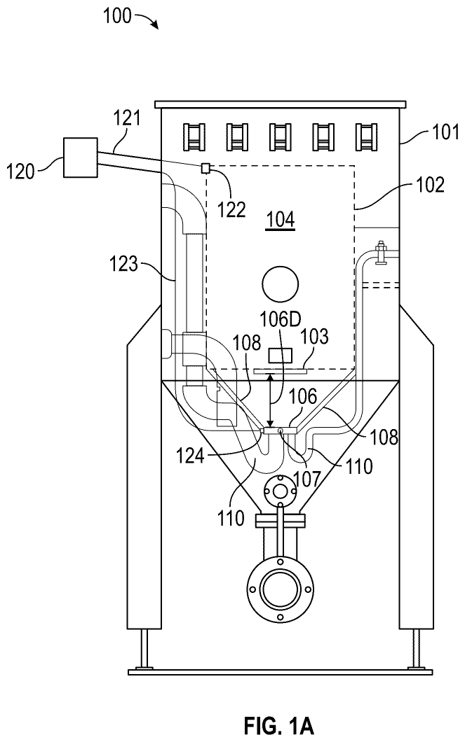

FIGS. 1A and 1B illustrate an exemplary bioreactor system 100. FIG. 1A illustrates a front view while FIG. 1B illustrates a side view of the bioreactor system 100. Bioreactor system 100 is operable to extract, produce, amplify, and/or recover metal from selection material 104. While the disclosure may refer to any of extract, produce, amplify, and/or recover, the end goal is to collect the desired metal that is present in the selection material 104. The metal can include, for example, metals, precious metals, rare earth metals, platinum group metals, gold, or any other desired metal. As used herein, the term “rare earth metals” or “RE” may refer to scandium (Sc), yttrium (Y), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and/or lutetium (Lu). As used herein, the term “precious earth metals” may refer to gold, silver, aluminum, rhenium, indium, platinum, gallium, germanium, ruthenium, rhodium, beryllium, palladium, osmium, iridium, tellurium, bismuth, platinum palladium, titanium, zinc, and/or zirconium.

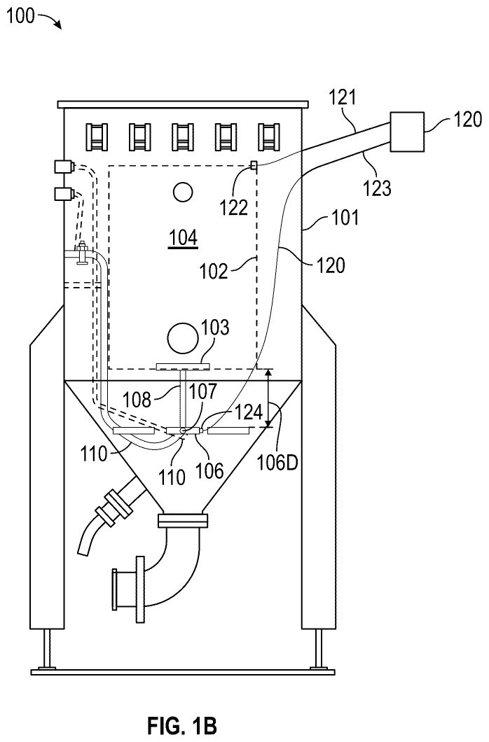

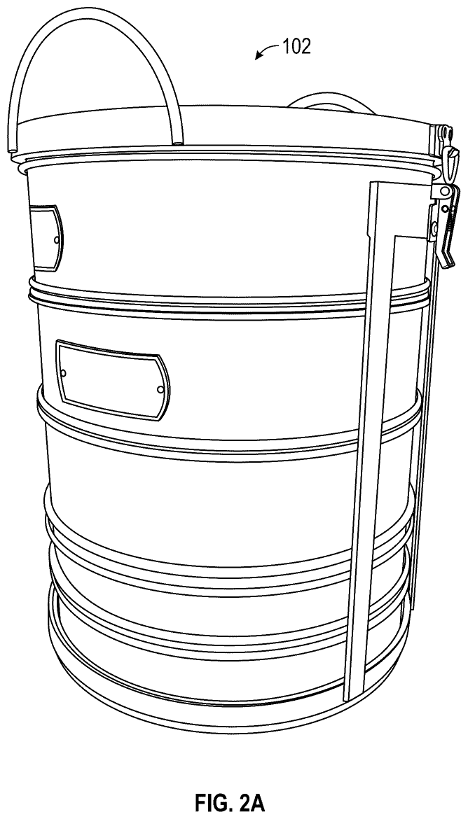

The bioreactor system 100 can include a housing 101 operable to contain one or more components of the bioreactor system 100. For example, as illustrated in FIGS. 1A-2B, a container 102 is disposed within the housing 101 of the bioreactor system 100. The container 102 is operable to contain the selection material 104 which includes the desired metal. The selection materials 104 can include, for example, water, ore, soil, rocks, substrates, nutrients for microbe, sugar sources, and/or any other suitable material. In some embodiments, the selection material is a geologic substrate surface (or volume) of sediment or rock where physical, chemical, and biological processes occur, such as the movement and deposition of sediment, the formation of bedforms, and the attachment, burrowing, feeding, reproduction, and sheltering of organisms. Non limiting examples of a geological substrate useful for the present disclosure include sandstone, limestone, shale, coal, chalk deposit formations, refractory rock ore (e.g., single, double and triple refractory rock ore). Additional solid substrates include, but are not limited, to an environmental sample collected from any terrestrial, aquatic or marine source such as soil, biofilms, sediments (e.g., coral or other marine sediments, aquifer sediments and the like), native metal rocks, and sludge residue. Additional substrates include but are not limited to animal manures, bauxite, base metals, calcium phosphate, calcium silicate, clays, silicates, aluminum oxide, diatomaceous earth, diammonium phosphate, erionite and zeolites, feldspar, flint, food wastes, granite, graphite, gypsum, humic and fulvic acids, marble, mica, molten rock and lava, monoammonium phosphate, potash, pumice, silica, slate, seaweed, talc and recycled electronics and commercial devices.

Also disposed in the selection material 104 is a microbe operable to recover the desired metal from the selection material. Microbes for use according to the disclosure are those capable to extract, produce and/or amplify precious metals and/or rare earth metals from a selection material. Non-limiting examples of suitable microbes include acidophilic archaea such as Sulfolobus metallicus and Metallosphaera sedula;mesophilic bacteria of the genera Acidithiobacillus or Leptospirillum ferrooxidans; Pyrococcus furiosus;thermoacidophilic archaeon Sulfolobus (Metallosphaera sedula); and Pyrobaculum islandicum.In an exemplary embodiment, a microbe for use according to the present disclosure comprises bacteria strain Thiomonas isabelensis (ECOAU001) as described U.S. Provisional Patent Application Ser. No. 63/196,509, the disclosure of which is herein incorporated in its entirety. ECOAU001 is a gram-negative bacterium, non-spore former that is capable of metabolizing simple and complex polymers as well as metals through heterotrophic and chemoheterotrophic biochemical pathways.

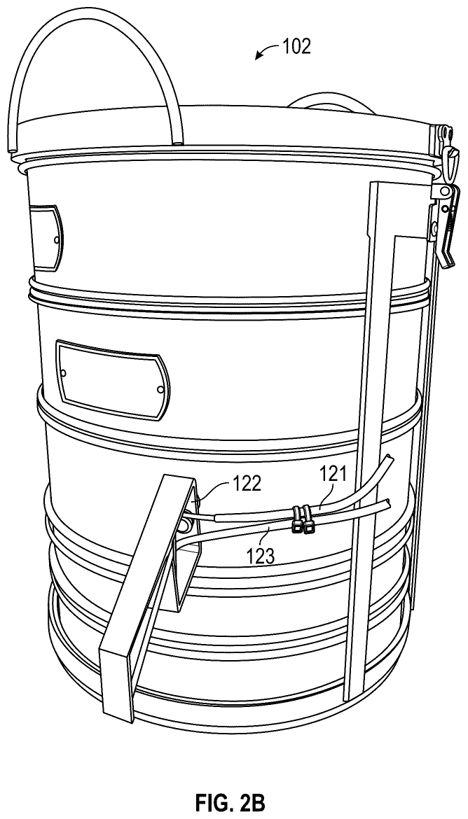

In at least one example, the microbe can be deposited into the selection material 104 via one or more input conduits 110. The microbe can be carried in water and/or air, and the microbe solution can be pumped through the input conduits 110 to be deposited in the container 102 and the selection material 104. Referring to FIGS. 1A, 1B, 3, and 4, the input conduits 110 can have an outlet 112 through which the microbe is pumped out of the input conduits 110. The outlet 112 can be aligned with a platform 106 through which the microbe passes through and/or passes by before entering the container 102. The platform 106 is operable to receive a metal sample 107 of the desired metal to be recovered. The metal sample 107 is operable to interact with the microbe and stimulate the microbe to recover the desired metal from the selection material 104. While FIGS. 1A-4 illustrate one platform 106 with one metal sample 107, in some examples, one platform 106 can include a plurality of metal samples 107. In some examples, a plurality of platforms 106 can be included, each receiving one or more metal samples 107. In at least one example, the metal samples 107 on one platform 106 can all be the same metal. By having the same metal as the metal samples 107, more interaction between the microbe and the metal sample 107 can further promote recovery of the desired metal in the selection material 104. In some examples, the metal samples 107 on one platform 106 can be different metals. Accordingly, multiple metals can be recovered within the selection material 104.

The platform 106 is in fluidic communication with the outlet 112 of the input conduit(s) 110. Accordingly, the fluid including the microbe is operable to pass through the platform 106, contact the metal sample 107, and be received in the selection material 104. In at least one example, the fluid including water, air, and the microbe can all be passed through one input conduit 110. In some examples, the water and microbe can be passed through one input conduit 110 while air can be introduced via another input conduit 110. The incorporation of air can create a dynamic and fast reaction with the microbe at the platform 106. Oxygen in the air can speed up the microbe metabolism to better guarantee a reaction between the microbe and the metal sample 107.

The fluid with the microbe can pass into the container 102 with the selection material 104 through a screen 103 which permits the fluid and microbe to enter the container 102 but prevents the selection material 104 or other material from leaving the container 102. As illustrated in FIGS. 1A and 1B, the platform 106 can be a distance 106D from the container 102 and/or the screen 103. In at least one example, the distance 106D can be about 0.5 inches to about 3 inches. In some examples, the distance 106D can be about 1 inches to about 2 inches. In some examples, the distance 106D can be about 1 inches. The distance permits the fluid with the microbe to contact the metal sample 107 in the platform 106, identify the desired metal, and then enter the selection material 104 to begin recovery of the desired metal.

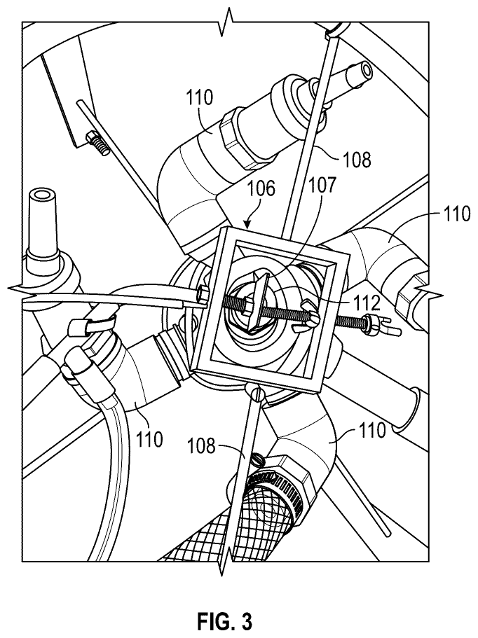

Referring to FIGS. 1A and 1B, a power source 120 can be coupled with the bioreactor system 100 to deliver electric pulses to the selection material 104 with the microbe and the metal sample 107. In at least one example, the power source 120 can be disposed external to the housing 101. In some examples, the power source 120 may be disposed in the housing 101. Referring also to FIG. 2B, the power source 120 can be coupled to a first line 121 with a first lead 122 that is operable to couple with an electrical conductor. As shown in FIGS. 1A, 1B, and 2B, the electrical conductor can include the container 102. The electrical conductor is operable to receive the transmit the electric pulses. The electrical conductor is in contact with at least a portion of the selection material 104 and the microbe therein such that the electric pulses are transmitted to the microbe in the selection material 104. Referring also to FIG. 4, the power source 120 can be coupled to a second line 123 with a second lead 124 that is operable to couple with the metal sample 107. In some examples, the second lead 124 can directly contact the metal sample 107. In some examples, the second lead 124 can be coupled to the platform 106.

The metal sample 107 and the metal conductor (e.g., the container 102) are electrically coupled to one another. For example, as illustrated in FIGS. 1A, 1B, and 4, the platform 106 is connected to a container frame 105, for example via sides 108. The container frame 105 is operable to receive the container 102. The container frame 105 and the sides 108 can all be electrical conductors such that electric pulses can pass between the metal sample 107 and the electrical conductor (e.g., the container 102). Accordingly, a closed circuit is achieved between the metal sample 107 and the electrical conductor (e.g., the container 102).

Delivering electric pulses can stimulate the microbe to improve recovery of the desired metal. In at least one example, the electric pulses can have wavelengths that correspond with a resonance of the desired metal. In some examples, the electric pulses can be between about 1 KiloHertz to about 1000 KiloHertz. For example, the electric pulses can be suitable for precious metals and/or rare earth metals. Accordingly, the electric pulses, passing through the metal sample 107 and the electrical conductor (e.g., the container 102), are able to provide specific wavelengths that correspond with the resonance of the desired metal to the microbe in the selection material. The microbe is then stimulated by the electric pulses to improve recovery of the desired metal, as the microbe can better recognize and be sensitized to the desired metal.

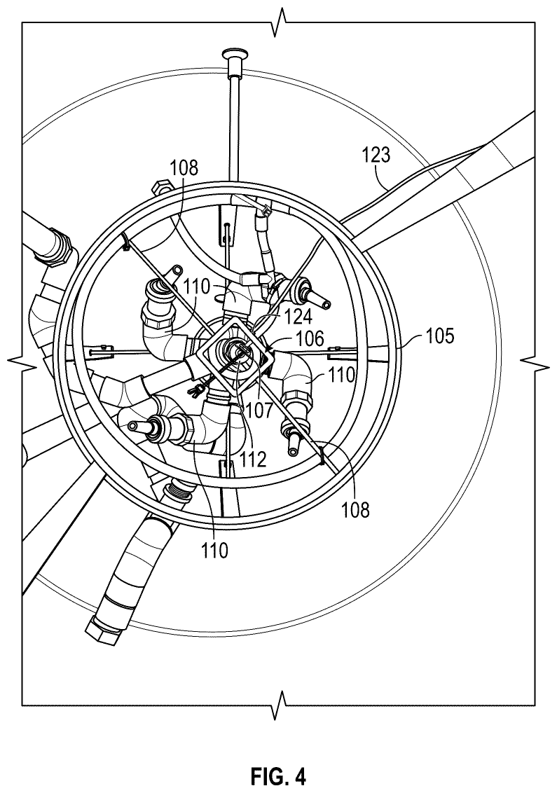

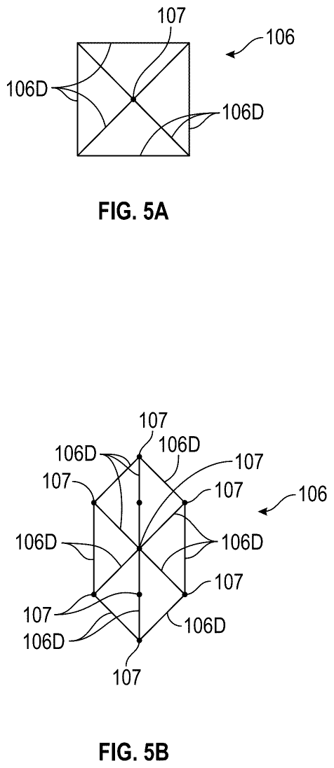

Referring to FIGS. 5A and 5B, the platform 106 can have different shapes and/or configurations to correspond with the metal sample 107. The platform 106 can have a shape that corresponds with the molecular structure of the metal sample 107. For example, as illustrated in FIG. 5A, the metal sample 107 is gold. The frame 1060 of the platform 106 is then configured to correspond with the molecular structure of gold. For example, the frame 1060 can have four outer sides forming a rectangular or square shape and diagonal sides that extend from the corners in towards the center at which the metal sample 107 is disposed. In another example, as illustrated in FIG. 5B, the frame 1060 can correspond with a metal crystal structure to correspond with the metal sample 107. The frame 1060 can have outer sides to form a hexagonal shape with inner sides that extend from each corner or junction in towards the center. A metal sample 107 can be disposed in the center. A plurality of metal samples 107 can also be disposed at different junctions and/or on sides of the frame 1060. By having a platform 106 that has a shape corresponding with the molecular structure of the metal sample 107, when the platform 106 receives the electric pulses, the shape of the platform 106 assists in further stimulating the microbe to focus on the desired metal. Accordingly, the microbe is able to recover a higher amount of the desired metal from the selection material 104.

The combination of the electric pulses and the microbe interacting with the metal sample 107 stimulates the microbe to yield a higher percentage of the desired metal available in the selection material 104. For example, the stimulated microbe (e.g., stimulated by the electric pulses and/or interaction with metal sample) may yield at least 5 to 10 times greater production than conventional methods and systems. For example, for precious metals, the stimulated microbe may achieve 10 to 100 times yield over conventional methods. For rare earth metals, the stimulated microbe may yield 10 times greater concentrations than conventional methods. In some examples, the combination of the electric pulses and the microbe interacting with the metal sample 107 can be referred to as a bioreactor stimulator. Additionally, the timeline to recover such a higher yield can be performed much quicker than conventional methods and systems. For example, the recovery of the desired metal can be completed in 1-2 days instead of months or years.

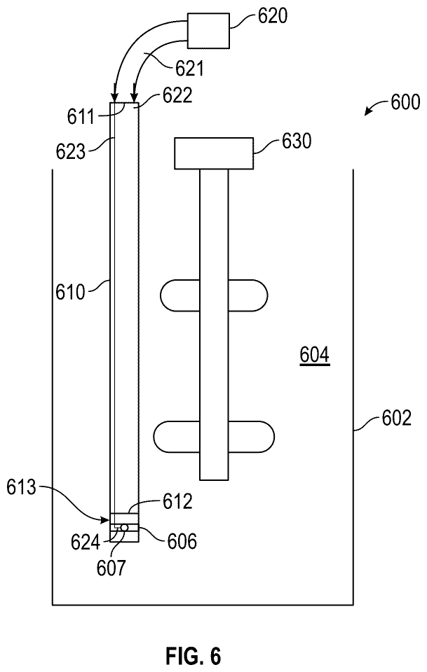

In some examples, the microbe can be deposited into the selection material 104 directly into the container 102. For example, the microbe can already be present in the selection material 104. The selection material 104 and/or the microbe can then receive the electric pulses to stimulate the microbe in the selection material 104 to recover the desired metal. For example, FIGS. 6-8 illustrate examples where the microbe can be disposed in a container, and a bioreactor stimulator can be provided to stimulate the recovery of the desired metal in the selection material in the container. Accordingly, the bioreactor stimulator is configured to be retrofit with conventional bioreactors and containers with selection material to stimulate the microbe to recover the desired metal with improved yield.

FIG. 6 illustrates a bioreactor stimulator 600 which is operable to be inserted into a container 602 that contains selection material 604 with the desired metal. In some examples, as illustrated in FIG. 6, the microbe can already be disposed in the selection material 604 before the bioreactor stimulator 600 is inserted into the container 602 and the selection material 604. The bioreactor stimulator 600 can include the platform 606 which coupled to and disposed proximate an end 612 of an electrical conductor 610. The platform 606 can be disposed in a platform region 613 which extends from the end 612 of the electrical conductor 610. The platform region 613 is configured so that the platform 606 and the metal sample 607 can be exposed to and contact the microbe in the selection material 604 when the bioreactor stimulator 600 is disposed in the container 602. In some examples, the walls of the platform region 613 may include one or more holes so that the microbe in the selection material 604 can gain access to and contact the metal sample 607.

In at least one example, an agitator 620 can be disposed in the container 602 to agitate and move the selection material 604 around inside the container 602. Accordingly, the microbe interacts with more selection material 604, and more microbe can interact with and contact the metal sample 607.

The electrical conductor 610, as illustrated in FIG. 6, can include a rod which is made of a material that can receive and transmit electrical pulses. While the rod 610 as illustrated in FIG. 6 is substantially cylindrical, the shape of the rod 610 can be rectangular, triangular, circular, or any other suitable shape to be inserted into the selection material 604. In some examples, as illustrated in FIG. 6, the ends 611, 612 of the electrical conductor 610 can be closed so that selection material 604 does not enter and clog up the inside of the electrical conductor 610.

The power source 620 can be connected to the first line 621 which has a first lead 622 that couples to the electrical conductor 610. In some examples, the first lead 622 can couple to the electrical conductor 610 towards or at the end 611 opposite the platform region 613 in relation to the electrical conductor 610. The second line 623 can have the second lead 624 which couples to the metal sample 607 and/or the platform 606. In at least one example, the power source 620 can be disposed external to the electrical conductor 610. For example, the power source 620 may include a wall outlet. In some examples, the power source 620 can be disposed within the electrical conductor 610. For example, the power source 620 may include a battery. The electrical conductor 610 and the metal sample 607 are operable to receive and transmit the electric pulses from the power source 620. As the electrical conductor 610 and the metal sample 607 form a closed circuit, the electric pulses pass from the metal sample 607 through the electrical conductor 610. The electric pulses pass through the electrical conductor 610 to the microbe in the selection material 604 to stimulate the microbe to recover the desired material.

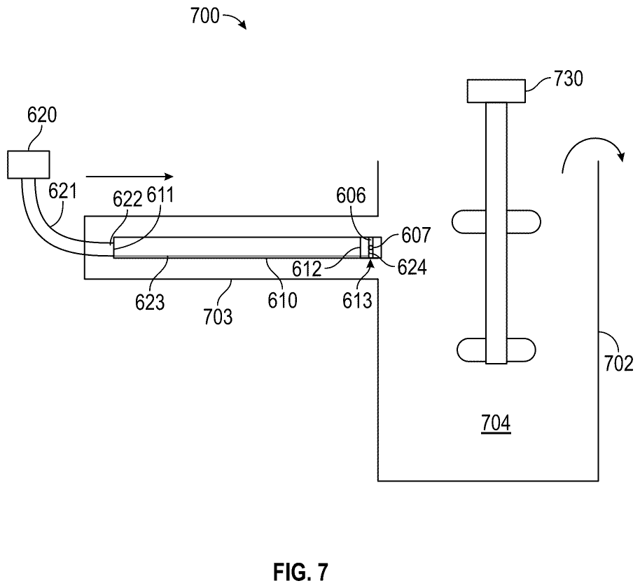

In some examples, as illustrated in FIG. 7, the bioreactor stimulator 600 can be disposed in an inlet conduit 703 of a bioreactor system 700. The platform region 613 can be disposed in the portion of the container 702 that contains the selection material 704. In at least one example, an agitator 720 can be disposed in the container 702 to agitate and move the selection material 704 around inside the container 702. Accordingly, the microbe interacts with more selection material 704, and more microbe can interact with and contact the metal sample 607. The bioreactor stimulator 600 can then stimulate the microbe with the metal sample 607 and the electric pulses to improve yield of the desired metal that is recovered from the selection material 704.

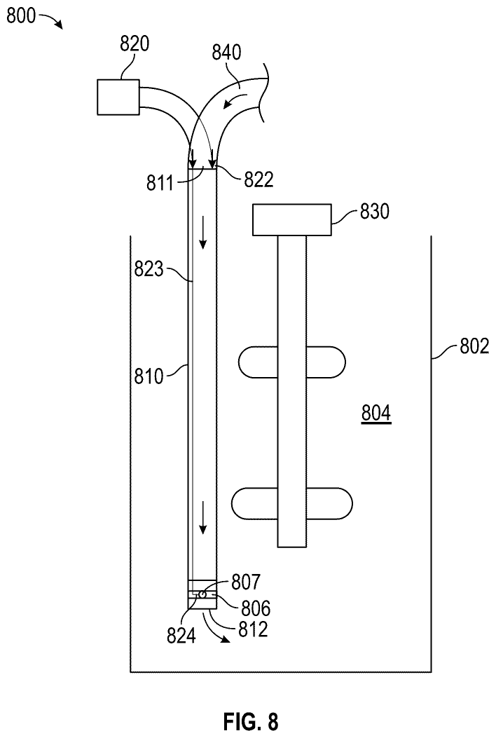

In some examples, as illustrated in FIG. 8, the bioreactor stimulator 800 can include an electrical conductor 810 which includes a tubular structure. The tubular structure 810 can be hollow and provide fluidic communication through the tubular structure 810 to the platform 806. The tubular structure 810 and the platform region 813 can be inserted into a container 802 that contains selection material 804.

An input conduit 840 can be fluidly coupled with an end 811 of the tubular structure 810. The input conduit 840 can provide fluid including the microbe (e.g., water, air, and microbe). For example, the input conduit 840 can be threadedly coupled with the end 811 of the tubular structure 810. The platform 806 can be in fluidic communication with the outlet 812 of the tubular structure 810, and the fluid including the microbe can be operable to pass through the electrical conductor 810, contact the metal sample 807, and be received in the selection material 804.

The power source 820 can be connected to the first line 821 which has a first lead 822 that couples to the electrical conductor 810. In some examples, the first lead 822 can couple to the electrical conductor 810 towards or at the end 811 opposite the platform region 813 in relation to the electrical conductor 810. The second line 823 can have the second lead 824 which couples to the metal sample 807 and/or the platform 806. In at least one example, the power source 820 can be disposed external to the electrical conductor 810. For example, the power source 820 may include a wall outlet. In some examples, the power source 820 can be disposed within the electrical conductor 810. For example, the power source 820 may include a battery. The electrical conductor 810 and the metal sample 807 are operable to receive and transmit the electric pulses from the power source 820. As the electrical conductor 810 and the metal sample 807 form a closed circuit, the electric pulses pass from the metal sample 807 through the electrical conductor 810. The electric pulses pass through the electrical conductor 810 to the microbe in the selection material 804 to stimulate the microbe to recover the desired material.

In at least one example, an agitator 830 can be disposed in the container 802 to agitate and move the selection material 804 around inside the container 802. Accordingly, the microbe interacts with more selection material 804, and more microbe can interact with and contact the metal sample 807.

Accordingly, with the bioreactor stimulator 600, 800 as illustrated in FIGS. 6-8, a portable bioreactor stimulator 600, 800 can be retrofitted into a conventional bioreactor system to improve yield of the desired metal. In at least one example, the bioreactor stimulator 600, 800 introduces the microbe to the selection material. In some examples, the selection material may already include the microbe. However, the bioreactor stimulator 600, 800 is retrofit with conventional bioreactor systems by introducing a metal sample to interact with the microbe and provide electric pulses to stimulate the microbe in the selection material to recover the desired metal.

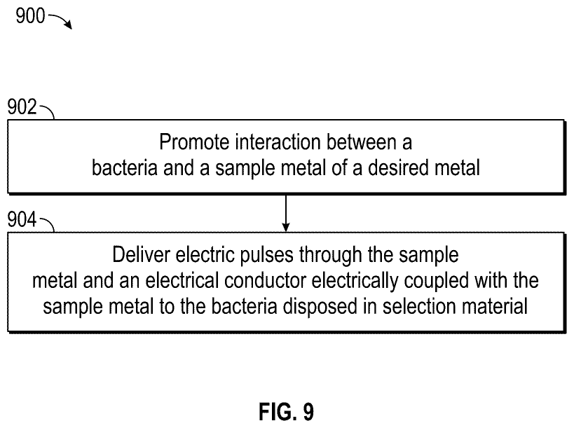

FIG. 9 is an example method 900 for tracking and/or tracing devices, in accordance with various aspects of the subject technology. The method 900 is provided by way of example, as there are a variety of ways to carry out the method. The method 900 described below can be carried out using the configurations illustrated in FIGS. 1A-8, for example, and various elements of these figures are referenced in explaining example method 900. Each block shown in FIG. 9 represents one or more processes, methods or subroutines, carried out in the example method 900. Furthermore, the illustrated order of blocks is illustrative only and the order of the blocks can change according to the present disclosure. Additional blocks may be added or fewer blocks may be utilized, without departing from this disclosure. The method 900 can begin at block 902.

At block 902, interaction is promoted between a microbe and a sample metal of a desired metal.

At block 904, electric pulses are delivered through the sample metal and an electrical conductor electrically coupled with the sample metal to the microbe disposed in selection material. The electric pulses stimulate the microbe to recover the desired metal in the selection material.

The electric pulses can have wavelengths corresponding with a resonance of the desired metal. The platform and the electrical conductor can be electrically coupled to form a closed circuit. Accordingly, the electric pulses transmit from the platform and electrical conductor to the microbe in the selection material to continue stimulating the microbe to recover the desired metal.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. A bioreactor stimulator comprising:
a platform operable to receive a metal sample of a desired metal, the metal sample operable to interact with microbe and stimulate the microbe to recover the desired metal from selection material;
an electrical conductor electrically coupled to the platform;
wherein the electrical conductor and the metal sample are operable to receive and transmit electric pulses,
wherein the electrical conductor is operable to transmit the electric pulses to the selection material to stimulate the microbe in the selection material to recover the desired metal.

2. The bioreactor stimulator of claim 1, wherein the electrical conductor includes a container containing the selection material and the microbe.

3. The bioreactor stimulator of claim 1, wherein the electrical conductor includes a rod.

4. The bioreactor stimulator of claim 1, wherein the electrical conductor includes a tubular structure.

5. The bioreactor stimulator of claim 4, wherein the platform is in fluidic communication with an outlet of the tubular structure; wherein fluid including the microbe is operable to pass through the electrical conductor, contact the metal sample, and be received in the selection material.

6. The bioreactor stimulator of claim 1, wherein the platform and the electrical conductor are electrically coupled to form a closed circuit.

7. The bioreactor stimulator of claim 1, wherein the platform has a shape corresponding with the molecular structure of the metal sample.

8. The bioreactor stimulator of claim 1, wherein the electric pulses have wavelengths corresponding with a resonance of the desired metal.

9. A bioreactor system comprising:
a container containing selection material and microbe, the microbe operable to recover a desired metal from the selection material;
a platform operable to receive a metal sample of a desired metal, the metal sample operable to interact with the microbe and stimulate the microbe to recover the desired metal from the selection material;
an electrical conductor electrically coupled to the platform;
wherein the electrical conductor and the metal sample are operable to receive and transmit electric pulses,
wherein the electrical conductor is operable to transmit the electric pulses to the microbe in the selection material to stimulate the microbe to recover the desired metal from the selection material.

10. The bioreactor system of claim 9, wherein the electrical conductor includes a container containing the selection material and the microbe.

11. The bioreactor system of claim 9, wherein the electrical conductor includes a rod, wherein the electrical conductor and the platform are operable to be removably inserted into the container and/or an inlet conduit for the container.

12. The bioreactor system of claim 9, wherein the electrical conductor includes a tubular structure, wherein the electrical conductor and the platform are operable to be removably inserted into the container and/or an inlet conduit for the container.

13. The bioreactor system of claim 12, wherein the platform is in fluidic communication with an outlet of the tubular structure; wherein fluid including the microbe is operable to pass through the electrical conductor, contact the metal sample, and be received in the selection material.

14. The bioreactor system of claim 9, wherein the platform and the electrical conductor are electrically coupled to form a closed circuit.

15. The bioreactor system of claim 9, wherein the platform has a shape corresponding with the molecular structure of the metal sample.

16. The bioreactor system of claim 9, wherein the electric pulses have wavelengths corresponding with a resonance of the desired metal.

17. The bioreactor system of claim 9, wherein an agitator disposed in the container is operable to agitate the selection material and the microbe.

18. A method comprising:
promoting interaction between a microbe and a metal sample of a desired metal; and
delivering electric pulses through the metal sample and an electrical conductor electrically coupled with the metal sample to the microbe disposed in selection material, the electric pulses stimulating the microbe to recover the desired metal in the selection material.

19. The method of claim 18, wherein the electric pulses have wavelengths corresponding with a resonance of the desired material.

20. The method of claim 18, wherein the platform and the electrical conductor are electrically coupled to form a closed circuit.
